(19) United States
(12) Patent Application Publication
(10) Pub. No.: US 2008/0167636 A1
(43) Pub. Date: Jul. 10, 2008

(54) Affinity Diaper

(76) Inventor: Brooke J. Saucier, Evanston, IL (US)

Correspondence Address: CARDINAL LAW GROUP, Suite 2000, 1603 Orrington Avenue, Evanston, IL, 60201, US

(21) Appl. No.: 11/621,450

(22) Filed: Jan. 9, 2007

(51) Int. Cl. A61F 13/49; B41M 3/00

(52) U.S. Cl. 604/385.101; 101/483

(57) ABSTRACT

A diaper includes an inner layer configured to allow fluid flow and an outer layer configured to prevent fluid flow, at least one material to absorb fluid within the inner layer and outer layer, and a first affinity image disposed on the outer layer. The diaper further includes a second affinity image disposed adjacent the inner layer, wherein the second affinity image is an antagonist image to the first affinity image.

### FIELD OF THE INVENTION

This invention generally relates to the art of diapers.

### BACKGROUND OF THE INVENTION

People with limited excretory control, such as the infirm and infants, wear diapers. Frequently these diapers include at least one image on the exterior of the diaper. The image often help determine a “front/back” and/or a logo or image intended to increase the saleability of the diaper. However, the images often fail to convey deeply held affinities of the parents of the baby.

The present invention advances the art.

### BRIEF DESCRIPTION OF THE SEVERAL VIEWS OF THE DRAWINGS

FIG. 1 illustrates one aspect of a diaper in accordance with one aspect of the invention;

FIG. 2 illustrates one aspect of a diaper in accordance with one aspect of the invention; and

FIG. 3 illustrates one aspect of a diaper in accordance with one aspect of the invention.

### DETAILED DESCRIPTION

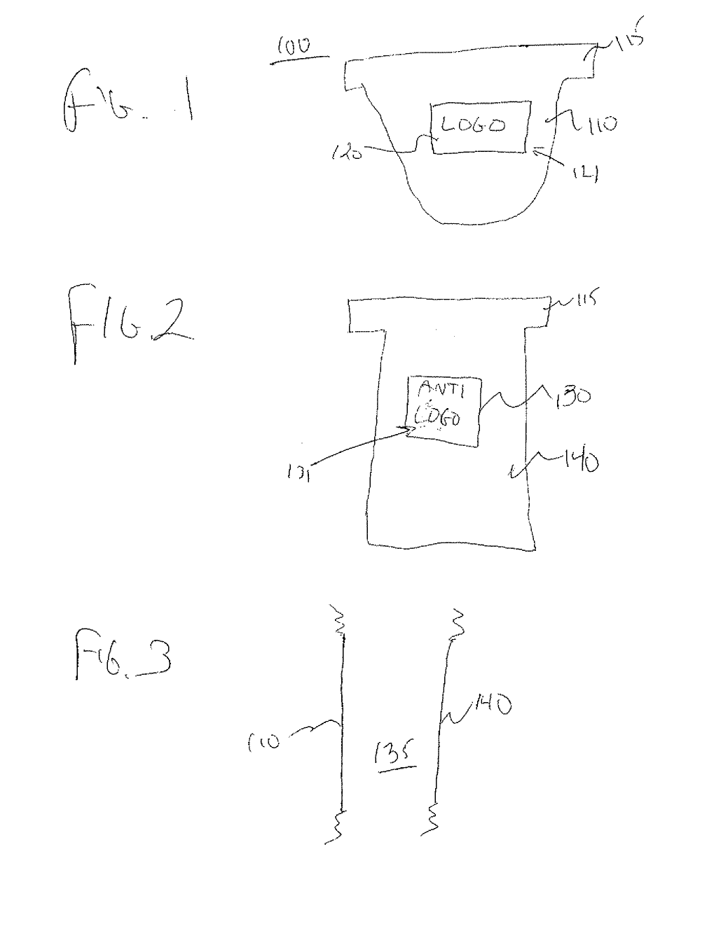

FIG. 1 illustrates a front view of one embodiment of a diaper 100 in accordance with one aspect of the invention. Diaper 100 includes outer layer 110 and straps 115 for affixing the diaper 100 in position. In addition, first affinity image 120 is disposed on the outer layer 110 so that first affinity image 120 is visible while the diaper is being worn. In one embodiment, the outer layer 110 is at least partially translucent.

FIG. 2 illustrates a top view of the diaper 100. Diaper 100, as seen in FIG. 2 further includes second affinity image 130 and inner layer 140. Second affinity image 130 is an antagonist image to the first affinity image 120. For example, first affinity image 120 is associated with a first sports team and second affinity image 130 is associated with a second, and rival, sports team. For example, if first affinity image 120 is associated with the University of Michigan, second affinity image 130 is associated with The Ohio State University. In another example, first affinity image 120 is associated with the Boston Red Sox and second affinity image 130 is associated with the New York Yankees. In another example, second affinity image 130 is selected as an image anathema to the first affinity image 120. In one embodiment, the inner layer 140 is at least partially translucent.

FIG. 3 illustrates a side cross sectional view of diaper 100. Diaper 100 includes inner layer 110 and outer layer 140 separated by an absorbent material 135. Absorbent material 135 is configured to absorb liquid or other fluid. In one embodiment, outer layer 140 is configured to allow fluid to flow into the absorbent material 135 and prevent fluid flow out of the material. The first affinity image and second affinity image can be disposed on an outer surface of the outer layer and inner layer, respectively, or between the material 135 and the appropriate layer. In embodiments where the images are not exterior to the layer, the layer is at least partially translucent to enable viewing of the image from outside the diaper. For example, the first affinity image and second affinity image can be printed directly on the diaper, on an insert placed within the diaper, and/or on a patch to be temporarily or permanently attached, such as with a hook-and-loop fastener, adhesive, or the like, to a diaper. In one embodiment, the ink used for the image is selected for contact with skin. In another embodiment, the ink is hypoallergenic.

In one embodiment, the first affinity image 120 is attached to a first affinity image zone 121 and the second affinity image is attached to a second affinity image zone 131. A plurality of affinity images, such as a set of images associated with each team within a provided group (i.e. all Major League Baseball teams, all Big Ten athletic conference teams, etc.) are provided and a user can select which affinity images to use as the first affinity image and second affinity image. In yet another embodiment, blank image receiving pieces are distributed, and a user can imprint each image receiving piece with a desired image.

The present invention may be embodied in other specific forms without departing from its spirit or essential characteristics. The described embodiments are to be considered in all respects only as illustrative and not restrictive.

1. A diaper comprising:
an inner layer configured to allow fluid flow and an outer layer configured to prevent fluid flow;
at least one material to absorb fluid within the inner layer and outer layer;
a first affinity image disposed on the outer layer; and
a second affinity image disposed adjacent the inner layer, wherein the second affinity image is an antagonist image to the first affinity image.

2. The diaper of claim 1 wherein the second affinity image is disposed upon the inner layer.

3. The diaper of claim 1 wherein the second affinity image is disposed between the outer layer and material.

4. The diaper of claim 3 wherein the outer layer is at least partially translucent.

5. The diaper of claim 1 wherein the outer layer is configured to allow fluid to flow into the material and to prevent fluid flow out of the material.

6. A diaper system comprising:
an outer layer including a first affinity image zone;
an inner layer including a second affinity image zone; and
a plurality of images configured to attach to one of the first affinity image zone and second affinity image zone.

7. The diaper of claim 6 wherein the outer layer is configured to allow fluid to flow into the material and to prevent fluid flow out of the material.

8. The diaper of claim 1 wherein the first affinity image is associated with a first sports team, and wherein the second affinity image is associated with a second sports team, and wherein the first sports team and second sports teams are rival sports teams.

9. The diaper system of claim 6 wherein the first affinity image is associated with a first sports team, and wherein the second affinity image is associated with a second sports team, and wherein the first sports team and second sports teams are rival sports teams.

10. The diaper of claim 1 wherein the second affinity image is anathema to the first affinity image.

11. The diaper system of claim 6 wherein the second affinity image is anathema to the first affinity image.

12. The diaper system of claim 6 wherein the plurality of images comprise a set of images associated with each team within a provided group.

13. The diaper system of claim 6 wherein the plurality of images comprise a blank image receiving piece configured to receive an imprinted desired image.

14. A method of providing a diaper, the method comprising:
providing a diaper including an outer layer including a first affinity image zone and an inner layer including a second affinity image zone;
determining a group;
determining a plurality of affinity images, each affinity image associated with a team within the determined group;
imprinting each of the plurality of affinity images on an image receiving piece;
attaching one of the affinity images to the first affinity image zone; and
attaching one of the affinity images to the second affinity image zone.
